(19) United States
(12) Patent Application Publication
(10) Pub. No.: US 2023/0355923 A1
(43) Pub. Date: Nov. 9, 2023

(54) INTRODUCER SHEATH WITH PEEL AWAY PERFUSION APERTURE

(71) Applicant: ABIOMED, Inc., Danvers, MA (US)

(72) Inventors: Jin Kwang Kim, Danvers, MA (US); Glen R. Fantuzzi, Danvers, MA (US)

(73) Assignee: ABIOMED, Inc., Danvers, MA (US)

(21) Appl. No.: 18/142,227

(22) Filed: May 2, 2023

Related U.S. Application Data
(60) Provisional application No. 63/338,207, filed on May 4, 2022.

(51) Int. Cl. A61M 25/00; A61M 25/01

(52) U.S. Cl. CPC A61M 25/005; A61M 25/01

(57) ABSTRACT

A perfusion sheath with covered perfusion apertures. The covers each have a pull affixed thereto. The pull is used to remove a perfusion aperture cover from a perfusion aperture when the perfusion sheath is deployed in a patient. The perfusion sheath is deployed in the patient through an arteriotomy. When deployed the pull or pulls extend out of the arteriotomy. Exerting a tension on the pulls causes the pull to tear away the perfusion aperture cover from the perfusion aperture. The perfusion sheath has a plurality of perfusion apertures, each positioned to provide limb perfusion, when the cover is removed for different depths of arteriotomy. Once the perfusion sheath is deployed, the pull is tensioned to remove the cover from a selected perfusion aperture that will provide limb perfusion for the depth of the arteriotomy.

### CROSS-REFERENCE TO RELATED APPLICATIONS

This application claims the benefit of and priority from U.S. Provisional Application No. 63/338,207, which was filed on May 4, 2022, and which is incorporated by reference herein.

### BACKGROUND

Intravascular medical devices such as percutaneous pumps (e.g. the Impella 2.5™ system by Abiomed, Inc., Danvers, Massachusetts), catheters, guidewires, balloon angioplasty catheters, delivery sheaths, and implant delivery systems are commonly used during minimally invasive procedures in the cardiovascular, cerebrovascular and peripheral vascular systems. Such medical devices can be introduced into a patient in various ways. Blood pump assemblies are introduced surgically or percutaneously during a cardiac procedure through the vascular system. Such intravascular procedures are minimally invasive. In one common approach, pump assemblies are inserted by a catheterization procedure through the femoral artery using a sheath, such as a peel-away introducer sheath. The femoral artery is commonly used in such procedures primarily due to ease of access. The sheath is inserted into the femoral artery through an arteriotomy (access site in the artery) to create an insertion path for the pump assembly. A portion of the pump assembly is then advanced through an inner lumen of the introducer sheath and into the artery. Once the pump assembly has been inserted, the introducer sheath is removed, for example by being peeled away. A repositioning sheath can then be advanced over the pump assembly and into the arteriotomy.

The introducer sheath must be large enough in diameter to accommodate the intravascular device, e.g., the blood pump. To date, the smallest available heart pump is the Impella 2.5 percutaneous heart pump, which has a motor diameter of about 12 Fr. The sheath must therefore be at least 13 Fr in order to introduce the Impella 2.5 pump. Other pumps and devices are larger than Impella 2.5, for example the Impella CP pump with a motor diameter of about 14 Fr, and the Impella 5.0 pump with a motor diameter of about 21 Fr or larger, depending on the type of device. An even larger sheath would be needed for such devices. Unless otherwise indicated in context, or would be understood by one of ordinary skill in the art, terms such as “about” mean within ±20%.

### BRIEF SUMMARY

Described herein is a perfusion sheath for providing persistent distal limb perfusion. The perfusion sheath includes a sheath body comprising an exterior surface and a lumen therethrough, at least one perfusion aperture in the sheath body positioned between the proximal end of the sheath body to the distal end of sheath body, a removable perfusion aperture cover positioned over the at least one aperture in the sheath body, and a pull attached to the removable perfusion aperture cover. The lumen extends from a distal end of the sheath body to a proximal end of the sheath body. The perfusion aperture provides for fluid communication through the exterior surface of the sheath body.

Also described herein is a method for providing persistent distal limb perfusion with a perfusion sheath as described above. The method includes providing a perfusion sheath described above, creating a puncture through a skin of a patient, inserting the perfusion sheath through the puncture and into an artery of the patient through an arteriotomy, positioning the perfusion sheath within the artery for the persistent distal limb perfusion, determining a perfusion aperture to be opened from the plurality of perfusion apertures, and tensioning the pull corresponding to the determined perfusion aperture to peel away the perfusion aperture cover associated with the pull and open the determined perfusion aperture to provide the persistent distal limb perfusion.

### BRIEF DESCRIPTION OF DRAWINGS

The foregoing and other objects and advantages will be apparent upon consideration of the following detailed description, taken in conjunction with the accompanying drawings, in which like reference characters refer to like parts throughout, and in which:

FIG. 1 shows an isometric view of an illustrative sheath according to an embodiment of the present invention;

FIG. 2 shows a side cross-sectional view of the sheath of FIG. 1 positioned in a shallow femoral artery;

FIG. 3 shows a side cross-sectional view of the sheath of FIG. 1 positioned in a medium femoral artery;

FIG. 4 shows a side cross-sectional view of the sheath of FIG. 1 positioned in a deep femoral artery;

FIG. 5 shows a side cross-sectional view of a sheath positioned in a femoral artery;

FIG. 6 shows a side cross-sectional view of the sheath of FIG. 5 positioned in a femoral artery with a perfusion aperture cover peeled away from the sheath body;

FIG. 7 shows a partial cross-sectional view of the sheath of FIG. 1 with a portion of a pull attached to a perfusion aperture cover;

FIG. 8 shows a partial side cross-sectional view of the sheath of FIG. 1 with a pull detaching a perfusion aperture cover;

FIG. 9 shows a partial side cross-sectional view of the sheath of FIG. 1 with a perfusion aperture cover peeled away from the sheath body by the pull; and

FIG. 10 is a flow chart of a method for providing persistent distal limb perfusion using the sheath of FIG. 1.

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

To provide an overall understanding of the systems, method, and devices described herein, certain illustrative embodiments will be described. Although the embodiments and features described herein are specifically described for use in connection with a percutaneous heart pump system, it will be understood that the components and other features outlined below may be combined with one another in any suitable manner and may be adapted and applied to other types of medical devices such as stents, transcatheter aortic valve replacement (TAVR) delivery systems, cardiac therapy and cardiac assist devices, including balloon pumps, cardiac assist devices implanted using a surgical incision, and other vascular devices.

While existing introducer sheaths are generally functional for device insertion, they have drawbacks. For example, due to its size, the introducer sheath can block the cross sectional area of the artery, and thereby occlude the artery, which can drastically restrict down-stream blood flow. Restricted blood flow can be problematic for a number of reasons, including causing intermittent claudication, leg numbness/weakness and limb ischemia which may even result in loss of the limb. Blood flow blockage can also increase the risk of bleeding at the access site, as pressure in arteriotomy builds due the restricted blood flow past the introducer sheath.

Conventionally, limb ischemia is known as a “distal perfusion” issue in the sense that it often occurs downstream from the access site of the device. In the present disclosure, a “distal” element is the part of the element located farthest away from the clinician and “proximal” is the part of the element that is closest to the clinician. In the case of a sheath deployed inside a patient, the sheath end that is deployed inside the body of the patient is the “distal” end, whereas the handle or end held by the clinician located outside the patient is the “proximal” end.

For relatively large medical devices (e.g., with a maximum outer diameter equal to or greater than 12 Fr), the introducer sheath (or repositioning sheath) used in combination with the medical device has an outer diameter that is substantially similar in size to an inner diameter of the artery or vessel in which the sheath is positioned. This match between the outer diameter of the sheath and inner diameter of the artery or vessel prevents blood from flowing past the location of sheath insertion. That is, blood flow may be blocked at a location upstream of the sheath disposed in the arteriotomy and impeded from flowing to a location downstream of the arteriotomy through which the sheath is disposed. Moreover, for these relatively large medical devices, the introducer sheath (or repositioning sheath) used in combination with the medical device also requires a large access site, which may be difficult to close, or to control bleeding when inserting or withdrawing the sheath into/from the arteriotomy. Whether a relatively large or relatively small medical device is deployed, and, accordingly, whether an introducer sheath or repositioning sheath is considered relatively large, may depend on a particular patient's anatomy.

In general, the challenges noted above are more pronounced for long-term vascular procedures. Certain procedures in the catheter lab are short term and therefore present less risk of limb ischemia. For example, procedures that typically only require at most 4-5 hours, with physicians periodically checking for distal (limb) perfusion, pose less risk than longer procedures. However, even for short-term procedures, technicians may not adequately monitor a patient to confirm that the patient's limb has adequate perfusion, or miss warning signs of inadequate limb perfusion. Additionally, certain patients may be transferred to the ICU with introducer sheaths or repositioning sheaths remaining in the patient for longer periods of times ranging from 1-14 days. In such instances, the problems of vessel occlusion and associated limb ischemia, and access site bleeding may be exacerbated.

A known intervention to an occluded femoral artery would be a bypass (i.e., a femoral artery to femoral artery (i.e., fem to fem) bypass for arterial revascularization. The technique includes gaining access, usually with a small sheath (e.g. with a 4 Fr catheter) to the opposite leg of the main access site. Using a similar sheath to gain access to a portion of the same leg distal to the main access site, the two sheaths are connected together to allow perfusion to the femoral artery of the patient's main access leg using blood flow from the opposite leg of the patient. However such an invasive procedure comes with other risks, including difficulty in gaining access to the portion of the femoral artery distal to the access site in the blocked leg (due to lack of flow, pressure, pulsatility, or a combination of these), additional allocation of resources (time, use of materials and extra components), increased risk of infection, and other risks associated with multiple access sites. Another potential intervention would be to use Extracorporeal Membrane Oxygenation (ECMO) to perfuse the blood distal to the access site. However, ECMO intervention may present the same challenges as those presented by fem-to-fem bypass.

Another potential intervention to address an access site with undesirably large bleeding would be to use collagen or a metallic clip or suture to close the access site. However, such an intervention is usually unplanned and rushed, and proper placement may be difficult to visualize in view of the ongoing bleeding at the access site.

Accordingly, there exists a need for an improved sheath for delivering an intravascular device and for providing persistent perfusion of blood within a patient distal to the device.

The systems, methods and devices of the present disclosure provide an introducer sheath for insertion into a blood vessel (e.g., an artery such as the femoral artery) of a patient. The sheath comprises a lumen configured for the passage of a medical device. The sheath is also configured to allow a flow of fluid within the artery without being substantially blocked from reaching areas of the patient downstream of the sheath, which will be described in greater details below.

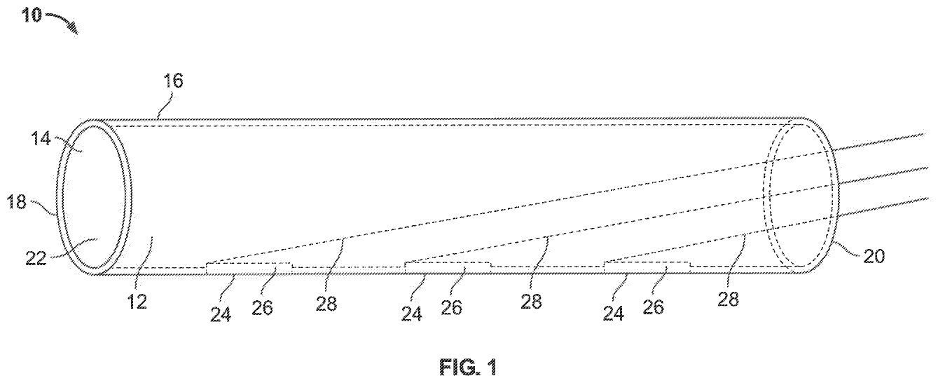

FIG. 1 shows an isometric view of an illustrative sheath 10 including one or more peel away perfusion apertures in accordance with one aspect of the present technology. The sheath 10, which may be an introducer sheath, a peel-away sheath, etc.) is suitable for insertion into an arteriotomy of a patient, which may be formed in a femoral artery of the patient. The sheath 10 comprises a sheath body 12 that has an inner surface 14 and an outer surface 16 and extends between a distal end 18 and a proximal end 20. As illustrated in FIG. 1, the sheath body 12 is tubular with an approximately circular cross section. As used herein, “approximately circular” is a shape that, to an observer, would appear more circular than some other shape. It will be appreciated that other designs and configurations could be used for the sheath body 12 as deemed suitable for its application.

The sheath body 12 comprises a lumen 22 extending between the distal end 18 and the proximal end 20 of the sheath body 12. The lumen 22 is open for the passage of a medical device such as a percutaneous blood pump (not shown). An example of such a percutaneous pump is the Impella 2.5° system (Abiomed, Inc., Danvers, Massachusetts). Such a pump generally may have a catheter body with a pump head at a distal end of the catheter body and a handle at a proximal end of the catheter body. It will be understood that other percutaneous or intravascular medical device can be used in conjunction with the present disclosure for delivering such a device through the sheath 10.

The lumen 22 of the sheath body 12 has an approximately circular cross section having a diameter. The lumen 22 is designed and dimensioned such that the sheath 10 is able to be inserted and to traverse within an artery (e.g., femoral artery), i.e., the diameter of lumen 22 is suitably smaller than the diameter of the artery. In addition, the lumen 22 is dimensioned such that a medical device is able to traverse the length of the interior of the lumen 22, i.e., the diameter of lumen 22 is suitably larger than the largest diameter of the medical device that will traverse therethrough. Thus, the diameter of the lumen 22 needs only be marginally larger than the maximum cross-section of the medical device and marginally smaller than the diameter of the artery.

Referring again to FIG. 1, the sheath 10 further comprises at least one perfusion aperture 24 formed along the length of the sheath body 12. While a sheath having only one peel away perfusion aperture is contemplated, the sheaths described herein have multiple peel away perfusion apertures, which may provide additional advantages described hereinbelow. FIG. 1 illustrates a plurality of peel away perfusion apertures 24. Also illustrated is a plurality of perfusion aperture covers 26 for covering the plurality of perfusion apertures 24, each cover having a pull affixed thereto. The pulls are biocompatible and extend outside the arteriotomy. Biocompatible materials are well known to one skilled in the art and are not described in detail herein. The pulls 28 are provided to peel away a cover 26 from a selected perfusion aperture. The pulls 28 may be strings, wires, filaments, braids, laces, and ribbons, etc. or any suitable linkage that may be able to attach to or be attached to a respective perfusion aperture cover 26. The attached plurality of pulls 28 may extend either along the inside of the sheath body 12 or the outer surface 16 of the sheath body 12. As noted above, the pulls may extend outside of the body of the patient when the sheath 10 is deployed in the patient.

The introducer sheath is deployed with a perfusion aperture cover 26 placed on each of the perfusion apertures 24. As described in greater detail herein, once the sheath is positioned in the patient's vasculature, the pull 28 attached to one of the perfusion aperture covers 26 of a selected perfusion aperture 24 is pulled, thereby removing a perfusion aperture cover 26 to which the pull is attached from a selected perfusion aperture thereby opening a perfusion aperture 24 in a sidewall of the introducer sheath body 12. The plurality of perfusion apertures 24 are positioned laterally on the sidewall of the introducer sheath body 12 and are intentionally spaced (e.g., evenly spaced) apart along the length of the sheath body 12. While the distance between the perfusion apertures 24 may be any distance suitable for application of the sheath 10, the distance of about 2.5 cm between each perfusion aperture is an example of a distance that allows the user to position the introducer sheath such that one of the perfusion apertures will be positioned in the blood flow pathway to support the desired perfusion. Each of the plurality of perfusion apertures 24 may be formed in any shape such as circular, oval, or square and sized to allow blood to flow therethrough at an acceptable rate when the perfusion aperture cover is removed from a perfusion aperture. Each of the plurality of perfusion aperture covers 26 are configured to be peeled away (or removed) from the sheath body 12 to allow blood to flow therethrough, as will be described in greater detail below.

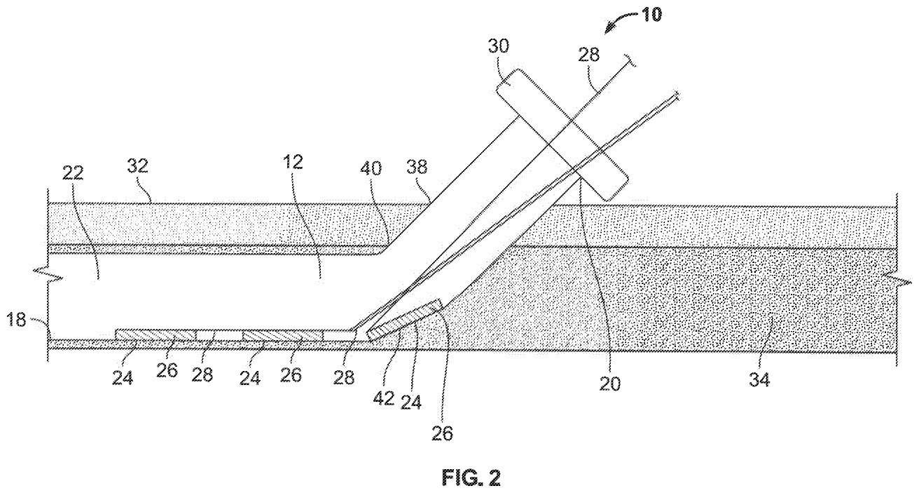

FIGS. 2-4 illustrate the sheath 10 inserted into an arteriotomy 40 of a patient with a sheath hub 30 sitting at the surface of the skin 32. When the sheath 10 is inserted in position, the distal end 18 of the sheath 10 is axially aligned with the femoral artery 34. Each figure shows the body tissue 32 of a patient and the femoral artery 34 with a skin insertion site 38 and the arteriotomy 40 where the sheath 10 is inserted into the femoral artery 34. The sheath body 10 bends as it enters the arteriotomy 40. When the sheath 10 is inserted into the artery 34, one or more perfusion apertures may be aligned within a blood flow pathway in the lumen of artery 34 while the remaining perfusion apertures are not aligned with the blood flow pathway in the lumen of the artery 34, but are disposed on the surface of the artery lumen or outside the artery lumen as shown in FIGS. 2-4.

After insertion of the sheath 10 into the artery 34, a clinician or other caregiver may peel away or remove the perfusion aperture cover 26 aligned with the blood flow pathway in the lumen in the artery 34 to open the perfusion aperture for providing persistent distal limb perfusion by allowing blood to flow out of the more proximal portion of the sheath. When the perfusion aperture is opened, blood flows from a location upstream of the sheath 10 through the distal end of the sheath, through the open perfusion aperture, and into the portion of the artery on the other side of the arteriotomy.

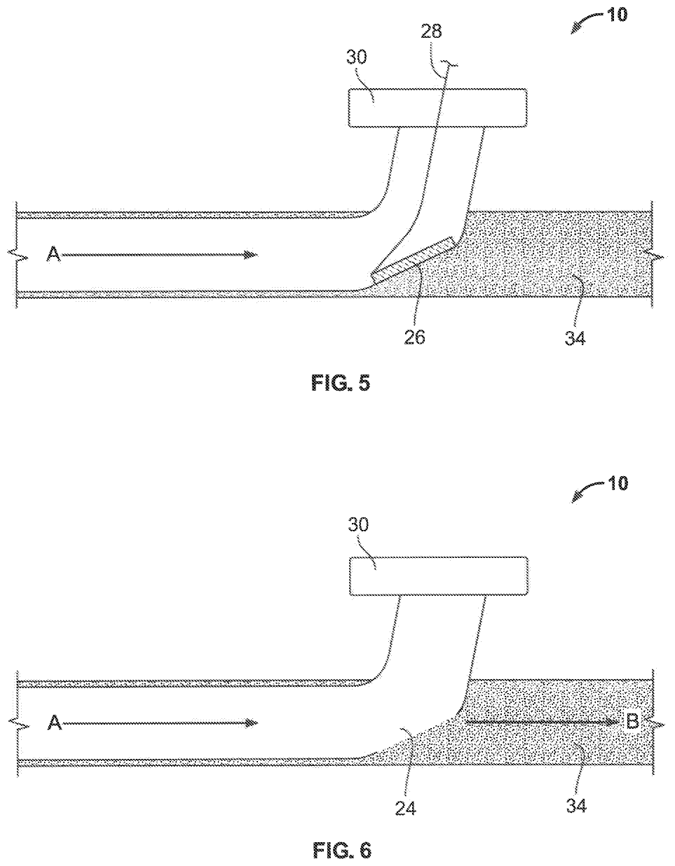

The plurality of perfusion apertures 24 are formed along the length of the sheath body 12 to meet patient's anatomic needs (i.e., the varying placements take into consideration patients' different anatomy for the depth of femoral artery relative to the skin surface). Specifically, covered perfusion apertures are placed so that a first perfusion aperture will align with a lumen of a shallow femoral artery, a second perfusion aperture will align with a lumen of a medium femoral artery, and a third perfusion aperture will align with a deep femoral artery. The depth is a length from the surface of the skin 32 and the interior lumen of the femoral artery 34. For example, as shown in FIGS. 2-4, depending on the depth of the femoral artery 34, one of the plurality of perfusion apertures 24 is located substantially along a bend 42 of the sheath 10 as it transitions from an insertion angle at the arteriotomy 40 to a position where the sheath 10 is positioned coaxially within the lumen of the artery 34. Thereafter, the perfusion aperture positioned at (or near) the bend 42 in the sheath body 12 (i.e., the perfusion aperture positioned with the lumen of the artery to permit blood to flow therethrough) may be opened (or uncovered) by withdrawing the pull 28/perfusion aperture cover 26 corresponding to the perfusion aperture at the bend 42 to allow blood to flow from the sheath lumen 22 to exit the sheath body 12 via the perfusion aperture and back into the artery 34, as indicated by arrows A and B in FIGS. 5 and 6, thereby maintaining flow of fluid in the artery 34. Specifically, the open perfusion aperture allows for blood to flow in the lumen 22 from the distal end 18 of the sheath 10 (the distal end of the sheath residing in the distal portion of the of the artery) through the open perfusion aperture to the opposite side of the arteriotomy for persistent distal limb perfusion and to prevent distal limb claudication.

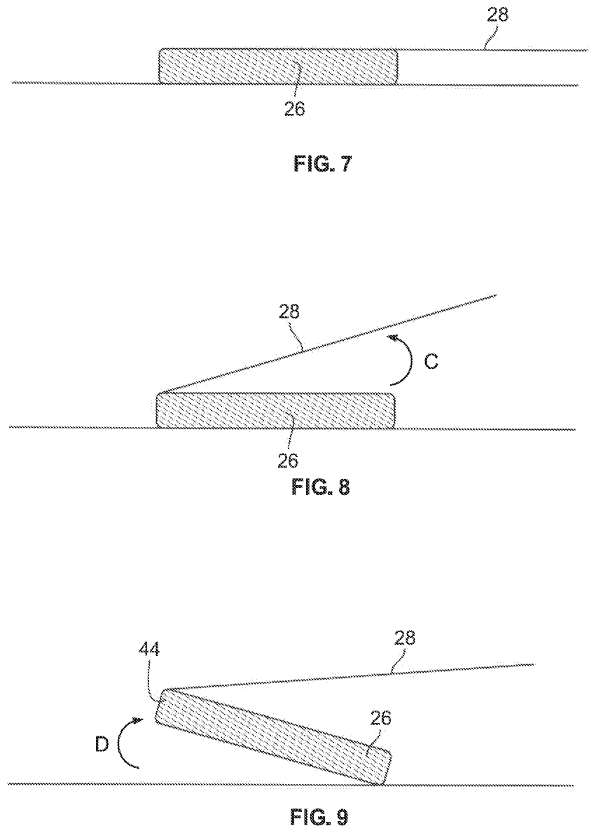

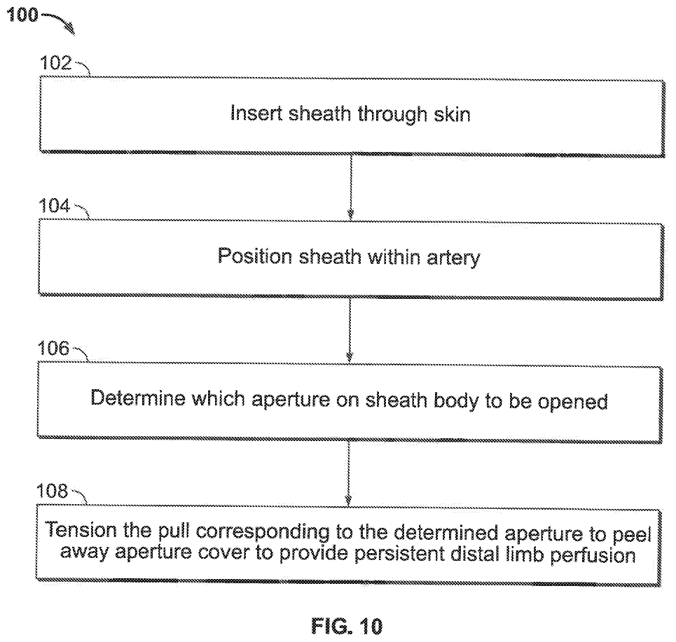

Each perfusion aperture 24 is initially covered with a peel-away perfusion aperture cover 26 with a portion of the corresponding pull 28 attached thereto as shown in FIG. 7. To open the selected perfusion aperture (i.e. the perfusion aperture positioned at the depth such that the aperture opening is aligned with direction of blood flow, which may be the perfusion aperture positioned most closely to the bend in the sheath body 12), the pull 28 attached to the perfusion aperture cover 26 corresponding to the selected perfusion aperture 24 is pulled such that it rises in the vertical direction indicated by arrow C at a first end of the perfusion aperture cover 26. The portion of the pull 28 at the first (proximal) end of perfusion aperture cover detaches therefrom, as shown in FIG. 8. Then, the pull 28 is pulled by a clinician to peel away and uncover the perfusion aperture 24. Specifically, when the pull 28 is tensioned, the perfusion aperture cover 26 is first detached from a second (distal) end 44 of the perfusion aperture cover 26. Then, with the further tensioning of the pull 28 (i.e., a pulling force), the perfusion aperture cover 26 is peeled away from the distal side of the perfusion aperture to the proximal side of the perfusion aperture as indicated by arrow D in FIG. 9.

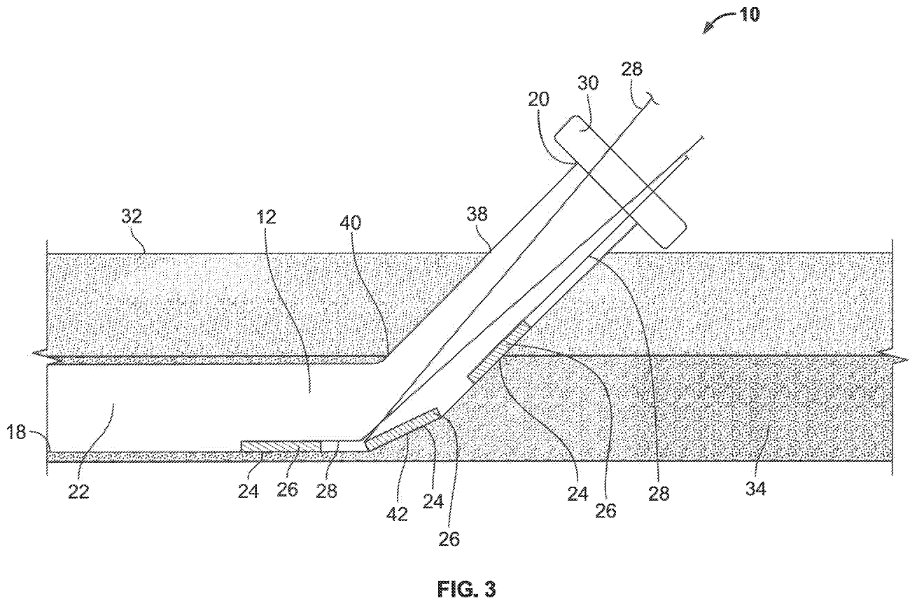

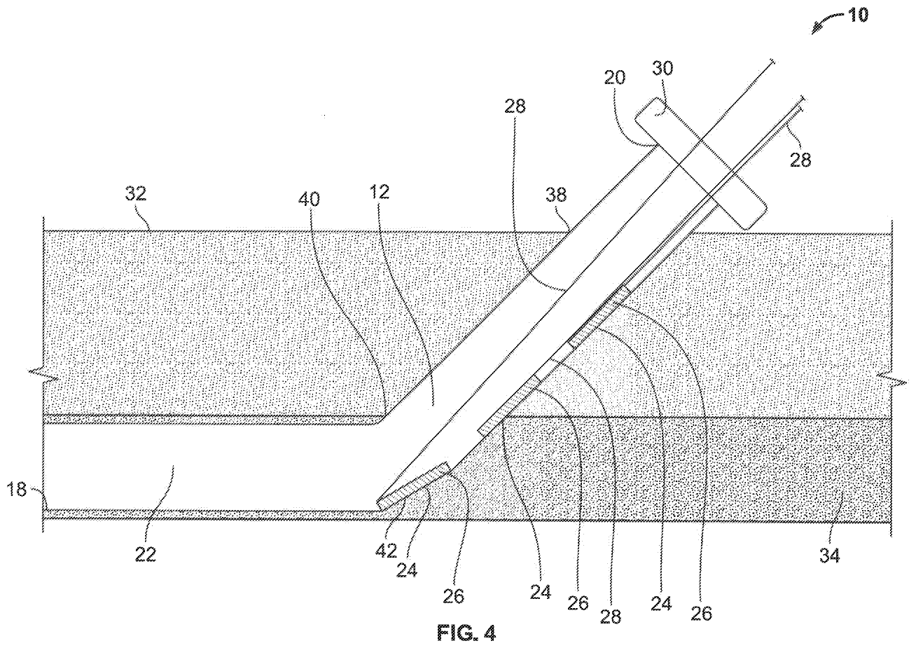

Referring again to FIGS. 2-4, the figures illustrate positioning of the sheath 10 in the shallow femoral artery, medium femoral artery, and deep femoral artery, respectively. In the shallow femoral artery (FIG. 2), where the depth from the surface of the skin 32 is less than 2 cm, the plurality of perfusion apertures 24 are arranged such that the perfusion aperture most proximal to the sheath hub 30 is positioned along the bend 42 of the sheath 10 as it transitions from the insertion angle at the arteriotomy 40 to a position where the sheath 10 lays in axial alignment with the lumen of the artery 34. In the medium femoral artery (FIG. 3), where the depth from the surface of the skin 32 is between 2 cm and 4.5 cm, the plurality of perfusion apertures 24 are arranged such that the middle perfusion aperture is positioned along the bend 42 of the sheath 10 as it transitions from the insertion angle at the arteriotomy 40 to a position where the sheath 10 lays in axial alignment with the lumen of the artery 34. Lastly, in the deep femoral artery (FIG. 4) where the depth from the surface of the skin 32 is greater than 4.5 cm, the plurality of perfusion apertures 24 are arranged such that the perfusion aperture farthest from the sheath hub 30 is positioned along the bend 42 of the sheath 10 as it transitions from the insertion angle at the arteriotomy 40 to a position where the sheath 10 lays in axial with the lumen of the artery 34. In some instances, ultrasound imaging may be used to determine a depth of the femoral artery for a particular patient. Alternatively or additionally, ultrasound imaging may facilitate the placement of one of the plurality of perfusion apertures 24 such that, when opened, the perfusion aperture will support persistent distal limb perfusion. For example, in some embodiments, the perfusion apertures and/or covers may be visible during ultrasonic imaging, and a user may be able to confirm that a particular perfusion aperture is properly positioned within the artery to support distal limb perfusion when the perfusion aperture is exposed by removing the cover.

While the sheath 10 having one perfusion aperture 24 (FIGS. 5 and 6) and sheath 10 having three perfusion apertures 24 (FIGS. 1-4 and 7-9) are shown in the present disclosure, any number of perfusion apertures can be formed along the length of the sheath body 12 of the sheath 10.

To assist a clinician with opening an appropriate perfusion aperture on the sheath body, each of the plurality of perfusion aperture covers 26 may be colored differently with each of the plurality of pulls 28 having the same color as its corresponding perfusion aperture cover 26. For example, referring to FIGS. 2-4, the perfusion aperture cover positioned closest to the sheath hub 30 and its corresponding pull may be blue whereas the perfusion aperture cover positioned farthest to the sheath hub 30 and its corresponding pull may be yellow.

The plurality of perfusion aperture covers 26 may be attached to the sheath 10 to cover the plurality of perfusion apertures 24 using various bonding techniques. These techniques prevent leaking through the perfusion aperture covers 26 and allow the perfusion aperture covers 26 to be peeled away (or removed) from the sheath body 12. As illustrated, the plurality of perfusion aperture covers 26 may be attached to the sheath body 12 with an adhesive designed to withstand high compressive forces but poor shear loads to enable the perfusion aperture covers 26 to be peeled away from the sheath body 12. In other aspects, the plurality of perfusion aperture covers may be thermally bonded (heat sealed) and perforated such that the perfusion apertures covers can be ripped away under high loads. Perforations may be applied by a thermal stamping or a laser etching operation.

The sheath 10 may be made of one or more materials having suitable properties for a desired application, including strength, weight, rigidity, etc. The sheath is made of a flexible plastic that is a biocompatible polymer such as thermoplastic polyurethane (TPU), nylon, polyether block amide (PEBA) expanded polytetrafluoroethylene (ePTFE), and blends thereof. In one particular aspect, the sheath 10 is made from one of TPU, Nylon, PEBA, and ePTFE.

FIG. 11. shows a method 100 for providing persistent distal limb perfusion using the sheath 10 in accordance with the present technology. In an initial procedure, at step 102, a clinician or other caregiver creates a puncture through the skin of a patient and percutaneously inserts the sheath 10 through the puncture site and into the artery of the patient through an arteriotomy. Additional steps may be performed by the clinician or caregiver between the puncture and the arteriotomy on one hand, and insertion of the sheath through the puncture and the arteriotomy on the other hand. For example, the clinician or other caregiver may use a guidewire, and successive dilators to gradually enlarge both the puncture and the arteriotomy sufficiently to accommodate the sheath 10, which can be an introducer sheath or a repositioning sheath. At step 104, the sheath 10 is advanced until its distal end 18 is positioned within the artery and the proximal end 20 of the sheath 10 is positioned outside the patient. After positioning the sheath 10, at step 106, the clinician determines which perfusion aperture is to be opened for persistent limb perfusion. Thereafter, at step 108, the pull 28 corresponding to the selected perfusion aperture 24 is pulled by the clinician to peel away (or remove) the cover 26 to which the pull 28 is attached from the sheath body 12 of the sheath 10. The perfusion aperture 24, now opened, allows blood to flow through the sheath body 12 of the sheath 10 from a location upstream of the arteriotomy to the downstream vasculature of the patient (downstream of the arteriotomy), thereby achieving persistent limb perfusion.

From the foregoing and with reference to the various figures, those skilled in the art will appreciate that certain modifications can also be made to the present disclosure without departing from the scope of the same. While several aspects of the disclosure have been shown in the figures, it is not intended that the disclosure be limited thereto, as it is intended that the disclosure be as broad in scope as the art will allow and that the specification be read likewise. Therefore, the above description should not be construed as limiting, but merely as exemplifications of particular aspects of the present technology.

### Exemplary Implementations

As already described, the perfusion sheath described herein may be implemented in various ways. In that regard, the foregoing disclosure is intended to include, but not be limited to, the systems, methods, and combinations and subcombinations thereof that are set forth in the following categories of exemplary implementations.

In a first aspect there is described a perfusion sheath having a sheath body with an exterior surface and a lumen therethrough, the lumen extending from a distal end of the sheath body to a proximal end of the sheath body. There is at least one perfusion aperture in the sheath body positioned between the proximal end of the sheath body to the distal end of sheath body, the perfusion aperture providing for fluid communication through the exterior surface of the sheath body. There is also a removable perfusion aperture cover positioned over the at least one aperture in the sheath body and a pull attached to the removable perfusion aperture cover.

In one aspect, when the sheath body is inserted into an arteriotomy, the pull has a length such that the pull will extend outside the arteriotomy. In a further aspect, the pull is selected from the group consisting of strings, wires, filaments, braids, laces, and ribbons. In yet a further aspect, the pull is a biocompatible material. According to any of the above aspects, the sheath body may have a plurality of perfusion apertures, each perfusion aperture provided with a removable cover affixed thereover and a pull affixed to each removable cover. According to any of the above aspects, the cover is affixed to the perfusion aperture using an adhesive or by heat sealing.

In any of the above aspects, the sheath may be made of a flexible plastic. In a further aspect, the flexible plastic may be a biocompatible polymer. The biocompatible polymer may be selected from the group consisting of thermoplastic polyurethane, nylon, polyether block amide, expanded polytetrafluoroethylene, and blends thereof.

In any of the above aspects, the pull may be affixed to the perfusion aperture cover from a proximal side of the perfusion aperture cover to a distal side of the perfusion aperture cover, the proximal side being positioned closer to the arteriotomy when the perfusion sheath is deployed in a patient than the distal side. In a further aspect, the pull may be lightly affixed to the perfusion aperture cover on the proximal side and more securely affixed to the perfusion cover on the distal side of the perfusion aperture cover, such that, when tensioned, the pull separates from the cover on the proximal side before being pulled away from the perfusion aperture on the distal side.

In a further aspect, the sheath may have three perfusion apertures, wherein each perfusion aperture is positioned a selected distance from the proximal end of the perfusion sheath. In yet a further aspect, the perfusion apertures are positioned such that, when the perfusion sheath is inserted into a patient, one of the perfusion apertures will be positioned along a bend of the perfusion sheath as the sheath transitions from an insertion angle at the arteriotomy to a position in axial alignment with a lumen of vasculature into which the perfusion sheath is inserted.

In a further aspect, the perfusion sheath may have a first perfusion aperture that has a selected position such that, when the perfusion sheath is inserted into the arteriotomy, the perfusion aperture is positioned about 2 cm or less beneath the surface of the arteriotomy. In yet a further aspect where there is a second perfusion aperture, that second perfusion aperture may have a selected position such that, when the perfusion sheath is inserted into the arteriotomy, the perfusion aperture is positioned greater than about 2 cm to about 4.5 cm beneath the surface of the arteriotomy. In those aspects where the perfusion sheath has a third perfusion aperture, that third aperture has a selected position such that, when the perfusion sheath is inserted into the arteriotomy, the perfusion aperture is positioned greater than about 4.5 cm beneath the surface of the arteriotomy.

In any of the above aspects, the pull may be a colored pull. In a further aspect, each pull may have a different color.

In another aspect, described herein is a method for providing persistent distal limb perfusion. According to the method a perfusion sheath is provided, the perfusion sheath having a sheath body that has an exterior surface and a lumen therethrough, the lumen extending from a distal end of the sheath body to a proximal end of the sheath body. The sheath body has a plurality of perfusion apertures in the sheath body positioned between the proximal end of the sheath body to the distal end of sheath body, where each perfusion aperture provides for fluid communication through the exterior surface of the sheath body. The apertures have a removable perfusion aperture cover positioned over each of the plurality of perfusion apertures in the sheath body with a pull attached to the removable perfusion aperture cover or covers. According to the method, an insertion site is prepared through the skin of a patient. The perfusion sheath is inserted through the puncture and into an artery of the patient through an arteriotomy. The perfusion sheath is positioned within the artery the persistent distal limb perfusion and it is thereafter determined if a perfusion aperture is to be opened and in those aspects where there are a plurality of perfusion apertures, one of the plurality of perfusion apertures is selected. A corresponding pull to the determined perfusion aperture is tensioned to peel away the perfusion aperture cover associated with the pull and open the determined perfusion aperture to provide the persistent distal limb perfusion.

In a further aspect, the opened perfusion aperture allows blood to flow through the sheath body of the sheath from a location upstream of the arteriotomy to downstream vasculature of the patient. According to the method, the perfusion sheath may be positioned within the artery for the persistent distal limb perfusion such that the proximal end of the sheath body is disposed outside the patient.

According to the above aspects of the method, the pull may be affixed to the perfusion aperture cover from a proximal side of the perfusion aperture cover to a distal side of the perfusion aperture cover, the proximal side of the perfusion aperture cover being positioned closer to the arteriotomy when the perfusion sheath is positioned in the patient than the distal side of the perfusion aperture cover. In a further aspect, the pull may be lightly affixed to the perfusion aperture cover on the proximal side of the perfusion aperture cover and more securely affixed to the perfusion aperture cover on the distal side of the perfusion aperture cover, such that, when tensioned, the pull separates from the perfusion aperture cover on the proximal side of the perfusion aperture cover before being pulled away from the perfusion aperture on the distal side of the perfusion aperture cover. In any of the above aspects, the pull may be a colored pull. In a further aspect, each pull has a different color.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

Although specific advantages have been enumerated above, various embodiments may include some, none, or all of the enumerated advantages.

Other technical advantages may become readily apparent to one of ordinary skill in the art after review of the following figures and description.

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise specifically noted, articles depicted in the drawings are not necessarily drawn to scale.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

1. A perfusion sheath comprising:
a sheath body comprising an exterior surface and a lumen therethrough, the lumen extending from a distal end of the sheath body to a proximal end of the sheath body;
at least one perfusion aperture in the sheath body positioned between the proximal end of the sheath body to the distal end of sheath body, the perfusion aperture providing for fluid communication through the exterior surface of the sheath body;
a removable perfusion aperture cover positioned over the at least one aperture in the sheath body; and
a pull attached to the removable perfusion aperture cover.

2. The perfusion sheath of claim 1, wherein, when the sheath body is inserted into an arteriotomy, the pull has a length such that the pull will extend outside the arteriotomy.

3. The perfusion sheath of claim 1, wherein the pull is selected from a group consisting of strings, wires, filaments, braids, laces, and ribbons.

4. The perfusion sheath of claim 1, wherein the pull is a biocompatible material.

5. The perfusion sheath of claim 1, wherein the sheath body comprises a plurality of perfusion apertures, each perfusion aperture provided with a removable cover affixed thereover and a pull affixed to each removable cover.

6. The perfusion sheath of claim 1, wherein the cover is affixed to the perfusion aperture using an adhesive or by heat sealing.

7. The perfusion sheath of claim 1, wherein the sheath is made of a flexible plastic.

8. The perfusion sheath of claim 7, wherein the flexible plastic is a biocompatible polymer.

9. The perfusion sheath of claim 8, wherein the biocompatible polymer is selected from the group consisting of thermoplastic polyurethane, nylon, polyether block amide, expanded polytetrafluoroethylene, and blends thereof.

10. The perfusion sheath of claim 2, wherein the pull is affixed to the perfusion aperture cover from a proximal side of the perfusion aperture cover to a distal side of the perfusion aperture cover, the proximal side being positioned closer to the arteriotomy when the perfusion sheath is deployed in a patient than the distal side.

11. The perfusion sheath of claim 10, wherein the pull is lightly affixed to the perfusion aperture cover on the proximal side and more securely affixed to the perfusion cover on the distal side of the perfusion aperture cover, such that, when tensioned, the pull separates from the cover on the proximal side before being pulled away from the perfusion aperture on the distal side.

12. The perfusion sheath of claim 5, wherein the sheath comprises three perfusion apertures, wherein each perfusion aperture is positioned a selected distance from the proximal end of the perfusion sheath.

13. The perfusion sheath of claim 12, wherein the perfusion apertures are positioned such that, when the perfusion sheath is inserted into a patient, one of the perfusion apertures will be positioned along a bend of the perfusion sheath as the sheath transitions from an insertion angle at the arteriotomy to a position in axial alignment with a lumen of vasculature into which the perfusion sheath is inserted.

14. The perfusion sheath of claim 13, wherein the perfusion sheath comprises a first perfusion aperture that has a selected position such that, when the perfusion sheath is inserted into the arteriotomy, the perfusion aperture is positioned about 2 cm or less beneath the surface of the arteriotomy.

15. The perfusion sheath of claim 13, wherein the perfusion sheath comprises a second perfusion aperture that has a selected position such that, when the perfusion sheath is inserted into the arteriotomy, the perfusion aperture is positioned greater than about 2 cm to about 4.5 cm beneath the surface of the arteriotomy.

16. The perfusion sheath of claim 13, wherein the perfusion sheath comprises a third perfusion aperture that has a selected position such that, when the perfusion sheath is inserted into the arteriotomy, the perfusion aperture is positioned greater than about 4.5 cm beneath the surface of the arteriotomy.

17. The perfusion sheath of claim 1, wherein the pull is a colored pull.

18. The perfusion sheath of claim 5, wherein each pull has a different color.

19. A method for providing persistent distal limb perfusion, the method comprising:
providing a perfusion sheath, the perfusion sheath comprising:
a sheath body comprising an exterior surface and a lumen therethrough, the lumen extending from a distal end of the sheath body to a proximal end of the sheath body;
a plurality of perfusion apertures in the sheath body positioned between the proximal end of the sheath body to the distal end of sheath body, each perfusion aperture providing for fluid communication through the exterior surface of the sheath body;
a removable perfusion aperture cover positioned over each of the plurality of perfusion apertures in the sheath body; and
a pull attached to the removable perfusion aperture cover;
creating a puncture through a skin of a patient;
inserting the perfusion sheath through the puncture and into an artery of the patient through an arteriotomy;
positioning the perfusion sheath within the artery for the persistent distal limb perfusion;
determining a perfusion aperture to be opened from the plurality of perfusion apertures; and
tensioning the pull corresponding to the determined perfusion aperture to peel away the perfusion aperture cover associated with the pull and open the determined perfusion aperture to provide the persistent distal limb perfusion.

20. The method of claim 19, wherein the opened perfusion aperture allows blood to flow through the sheath body of the sheath from a location upstream of the arteriotomy to downstream vasculature of the patient.

21. The method of claim 19, wherein the perfusion sheath is positioned within the artery for the persistent distal limb perfusion such that the proximal end of the sheath body is disposed outside the patient.

22. The method of claim 19, wherein the pull is affixed to the perfusion aperture cover from a proximal side of the perfusion aperture cover to a distal side of the perfusion aperture cover, the proximal side of the perfusion aperture cover being positioned closer to the arteriotomy when the perfusion sheath is positioned in the patient than the distal side of the perfusion aperture cover.

23. The method of claim 22, wherein the pull is lightly affixed to the perfusion aperture cover on the proximal side of the perfusion aperture cover and more securely affixed to the perfusion aperture cover on the distal side of the perfusion aperture cover, such that, when tensioned, the pull separates from the perfusion aperture cover on the proximal side of the perfusion aperture cover before being pulled away from the perfusion aperture on the distal side of the perfusion aperture cover.

24. The method of claim 19, wherein the pull is a colored pull.

25. The method of claim 24, wherein each pull has a different color.
